(19) United States
(12) Patent Application Publication
(10) Pub. No.: US 2009/0098136 A1
(43) Pub. Date: Apr. 16, 2009

(54) USE OF TNF RECEPTOR ANTAGONISTS FOR TREATING DRY EYE

(75) Inventors: Daniel A. Gamache, Arlington, TX (US); John M. Yanni, Burleson, TX (US); Martin B. Wax, Westlake, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/251,699

(22) Filed: Oct. 15, 2008

Related U.S. Application Data
(60) Provisional application No. 60/980,033, filed on Oct. 15, 2007.

(51) Int. Cl. A61K 39/395; A61P 27/02

(52) U.S. Cl. 424/141.1; 424/130.1

(57) ABSTRACT

Methods of treating dry eye by administering inhibitors of tumor necrosis factor α (TNFα) are disclosed.

### CROSS-REFERENCE TO RELATED APPLICATION

This application claims priority under 35 U.S.C. §119 to U.S. Provisional Patent Application No. 60/980,033 filed Oct. 15, 2007, the entire contents of which are incorporated herein by reference.

### FIELD OF THE INVENTION

The invention relates to the treatment of dry eye disorders. In particular, the invention relates to the use of certain tumor necrosis factor alpha (“TNFα”) inhibitors in the treatment of dry eye.

### BACKGROUND OF THE INVENTION

Dry eye, also referred to as keratoconjunctivitis sicca, is a common ophthalmological disorder affecting millions of persons each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid, may also lead to dry eye conditions. Transient symptoms of dry eye associated with refractive surgery have been reported to last in some cases from six weeks to six months or more following surgery.

Although it appears that dry eye may result from a number of unrelated pathogenic causes, all presentations of the complication share a common effect, that is the breakdown of the pre-ocular tear film, which results in exposure of the ocular surface, dehydration, and cytokine production resulting in many of the symptoms outlined above (Lemp, Report of the National Eye Institute/Industry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine, Tear film structure and dry eye, Contactologia, volume 20(4), pages 145-49 (1998); and Shine and McCulley, Keratoconjunctivitis sicca associated with meibomian secretion polar lipid abnormality, Archives of Ophthalmology, volume 116(7), pages 849-52 (1998).

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Although these approaches have met with some success, problems in the treatment of dry eye nevertheless remain, since the use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive.

Aside from efforts described above, which are directed primarily to the palliative alleviation of symptoms associated with dry eye, methods and compositions directed to treatment of the physiological conditions that cause such symptoms have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production.

Such efforts to treat the underlying causes of dry eye have focused on treating inflammation of the relevant ocular tissues and meibomian gland dysfunction. The use of various types of agents for such treatment of dry eye patients has been disclosed, including steroids (e.g., U.S. Pat. No. 5,958,912; Marsh et al., Topical nonpreserved methylprednisolone therapy for keratoconjunctivitis sicca in Sjogren syndrome, Ophthalmology, 106(4): 811-816 (1999); and Pflugfelder et al., U.S. Pat. No. 6,153,607), cytokine release inhibitors (Yanni, J. M.; et. al. WO 00/03705 A1), cyclosporine A (Tauber, J. Adv. Exp. Med. Biol. 1998, 438 (Lacrimal Gland, Tear Film, and Dry Eye Syndromes 2), 969), and mucosecretatogues, such as 15-HETE (Yanni et. al., U.S. Pat. No. 5,696,166).

TNFα is a major mediator of the inflammatory response, and has been implicated in many human diseases. Binding of TNFα to its cell surface receptor, TNF receptor-1 (“TNFR1”), activates a signaling cascade affecting a wide variety of cellular responses, including apoptosis and inflammation. TNFα is initially expressed as an inactive, membrane-bound precursor. Release of the active form of TNFα from the cell surface requires proteolytic processing of the precursor by TNFα converting enzyme/a disintegrin and metalloproteinase domain 17 (“TACE/ADAM 17”).

U.S. Pat. No. 6,428,787 (Tobinick et al.), U.S. Pat. No. 6,379,666 (Tobinick et al.), U.S. Pat. No. 6,177,077 (Tobinick et al.), U.S. Pat. No. 6,204,270 (Ron et al.), U.S. Patent Application Publication No. 2004/0126372 (Banerjee et al.), and International Application WO 00/27421 describe the possible involvement of TNFα in a number of eye disorders. Of the foregoing, only WO 00/27421 and US 2004/0126372 mention dry eye as among the eye disorders for which TNFα inhibitors may be useful. However, neither of these applications provides meaningful teaching relative to selection of a TNFα inhibitor or a concentration of a selected TNFα inhibitor effective for treating dry eye. In particular, it is shown herein that Pegsunercept, a monomeric soluble TNFR p55 receptor, is not effective for treating dry eye at clinically relevant concentration levels. In addition, as shown herein for the first time, dimeric soluble TNFα receptors and monoclonal antibodies specific for TNFα are effective for treating dry eye as demonstrated with in vivo dry eye models.

### SUMMARY OF THE INVENTION

The invention provides methods for the treatment of dry eye and other disorders that require restoring an intact ocular surface and normal tear function, including symptoms of dry eye associated with refractive surgery such as LASIK surgery. According to the methods of the invention, certain TNFα inhibitors are administered to a patient suffering from dry eye. The TNFα inhibitors are preferably administered topically to the eye.

Specific preferred embodiments of the invention will become evident from the following more detailed description of certain preferred embodiments and the claims.

### BRIEF DESCRIPTION OF THE DRAWINGS

FIG. 1 shows a graph depicting the effects of Enbrel® and Remicade® on tear breakup time measured on Day 3 of the dosing regimen.

FIG. 2 shows a graph depicting the effects of Enbrel® and Remicade® on corneal staining.

FIG. 3 shows a graph depicting the effects of Pegsunercept and vehicle controls on tear breakup time measured on Day 3 of the dosing regimen.

FIG. 4 shows a graph depicting the effects of Pegsunercept and vehicle controls on corneal staining.

### DETAILED DESCRIPTION OF THE INVENTION

According to the invention, inhibitors of “TNFα” are administered to a patient suffering from dry eye. The compounds suitable for use in the present invention inhibit the activity of TNFα by binding to TNFα at the ocular surface of a patient, thereby reducing the pro-inflammatory effects of TNFα associated with dry eye.

The term “TNFα inhibitor” includes any agent that can inhibit the activity of TNFα at an ophthalmically relevant concentration, and which is more potent (i.e. neutralizes TNFα to a greater extent) than a monomeric soluble TNFR p55 receptor, such as Pegsunercept. As used herein, an “ophthalmically relevant concentration” is less than 1.0% (w/w). Preferred TNFα inhibitors are soluble dimeric TNFα receptors, such as etanercept, which is a dimeric fusion protein of the extracellular ligand-binding portion of the human TNFα receptor (p75) linked to the Fc portion of human IgG1, and anti-TNFα antibodies, such as infliximab, which is a chimeric IgG1 monoclonal antibody that binds specifically to human TNFα.

According to the methods of the present invention, a composition comprising one or more of the specified TNFα inhibitors and a pharmaceutically acceptable carrier for topical ophthalmic administration or implantation into the conjunctival sac or anterior chamber of the eye is administered to a mammal in need thereof. The compositions are formulated in accordance with methods known in the art for the particular route of administration desired.

The compositions administered according to the present invention comprise a pharmaceutically effective amount of one or more of the specified TNFα inhibitors. As used herein, a “pharmaceutically effective amount” is one which is sufficient to reduce or eliminate signs or symptoms of dry eye. Preferably, compositions are intended to be administered topically to the eye in the form of eye drops or eye ointments, wherein the total amount of TNFα inhibitor will be about 0.001 to 1.0% (w/w). Preferably, the amount of TNFα inhibitor is about 0.01 to about 1.0% (w/w).

Preferably, the compositions administered according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions. Suspensions may be preferred for cytokine synthesis inhibitors which are sparingly soluble in water.

The compositions administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6.0-7.5.

Topical ophthalmic products may also be packaged in multidose form. Preservatives may thus be required to prevent microbial contamination during use. Suitable preservatives include: chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives. However, the ophthalmic compositions of the present invention are preferably preservative free and packaged in unit dose form.

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, 1-2 drops of such compositions will be administered one or more times per day. For example, the composition can be administered 2 to 3 times a day or as directed by an eye care provider.

A representative eye drop formulation is provided in Table 1 below.

TABLE 1

| Ingredient | Amount (% w/w) |
|---|---|
| TNFα inhibitor | 0.001-1.0 |
| Boric Acid | 0.25 |
| Sodium Chloride | 0.75 |
| Disodium Edetate | 0.01 |
| Polyquaternium-1 | 0.001 |
| NaOH/HCl | q.s., pH = 7.4 |
| Purified Water | q.s. 100% |

The above composition is prepared by the following method. The batch quantities of boric acid, sodium chloride, disodium edetate, and polyquaternium-1 are weighed and dissolved by stirring in 90% of the batch quantity of purified water. The pH is adjusted to 7.4.±0.0.1 with NaOH and/or HCl. The batch quantity of the TNFα inhibitor as a stock solution is measured and added. Purified water is added to q.s. to 100%. The mixture is stirred for five minutes to homogenize and then filtered through a sterilizing filter membrane into a sterile recipient.

All references cited in this application are expressly incorporated by reference herein for any purpose.

Unless otherwise required by context, singular terms used herein shall include pluralities and plural terms shall include the singular.

### EXAMPLES

The following examples, including the experiments conducted and results achieved are provided for illustrative purposes only and are not to be construed as limiting the invention.

### Example 1

### Effects of TNFα Inhibitors on Tear Break Up Time and Corneal Staining

To examine the effect of TNFα inhibition as a treatment for dry eye, three TNFα inhibitors were obtained and tested in clinically relevant concentrations for topical efficacy in the rabbit dry eye model using routine procedures. The tested agents represented various types of TNFα inhibitors, including a dimeric soluble TNFα receptor (etanercept), a monomeric soluble TNFα receptor (Pegsunercept), and a monoclonal anti-TNFα antibody (infliximab).

Enbrel® (AMGEN, Thousand Oaks, Calif. and Wyeth Pharmaceuticals, Madison, N.J.) was utilized as the source for the dimeric soluble TNFα receptor (etanercept), Remicade® (Centocor, Inc., Hersham, Pa.) was utilized as the source for the monoclonal anti-TNFα antibody (infliximab), and Pegsunercept was utilized as the monomeric soluble TNFα receptor. Enbrel® and Remicade® were obtained as powders and reconstituted in sterile saline at concentrations of 0.01%, 0.1% and 1.0%. Persunercept was used in a vehicle of 10 mM L-histidine, 2% L-glycine, and 1% sucrose in water.

Dry eye was induced in New Zealand white rabbits (approximately 2 kg) by eliciting bilateral inflammation of the lacrimal glands as previously described (Nagelhout et al., 2005, Journal of Ocular Pharmacology and Therapeutics, 21:139-148). Tear function was assessed by measuring tear breakup time (TBUT) daily for three days following the induction of dry eye. TBUT was determined by instilling 5 μL sodium fluorescein into the cul de sac and manually blinking the lids to distribute the fluorescein within the tear film. Under slit lamp observation, the eye was held open and the time to tear film breakup recorded. Efficacy was determined by comparing TBUT relative to pre-inflammation baseline values in drug- and vehicle-treated animals. In a separate group of animals, susceptibility to desiccation-induced corneal injury was assessed following the induction of lacrimal gland inflammation. Desiccation was initiated by placing the rabbits in a low humidity environment continuously for up to three days. Corneal injury was assessed by determining the uptake of the vital dye methylene blue. Under general anesthesia, the ocular surface was bathed in a 1% solution of methylene blue for five minutes and then washed. The animals were euthanized, eyes were excised and an 8-mm diameter section of cornea was isolated and extracted overnight. The concentration of extracted dye was determined spectrophotometrically (A₆₆₀). Protection of the cornea was indicated by a lesser uptake of dye in drug treated animals relative to that in vehicle treated rabbits. For both TBUT and corneal injury determinations, dosing (BID) was initiated 24 hours prior to inducing inflammation and was continued for the duration of the study; unless otherwise noted.

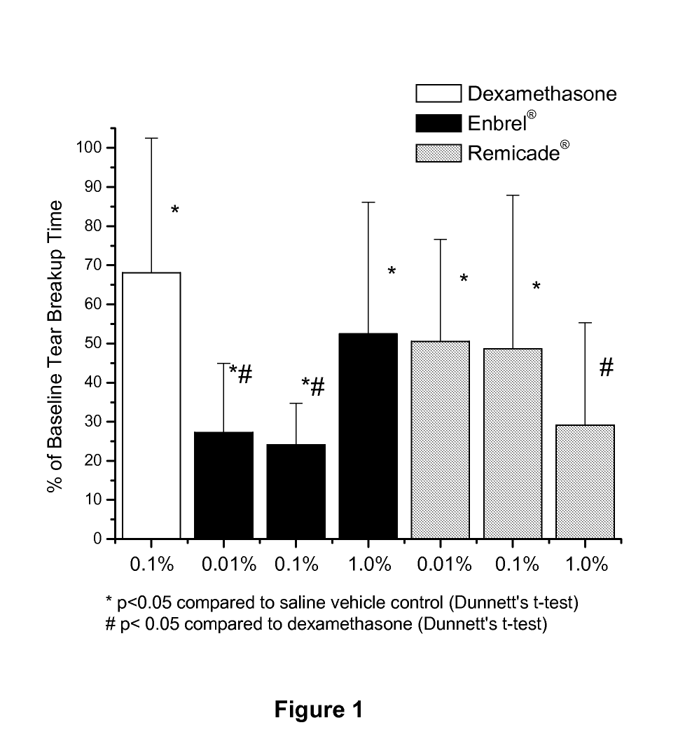

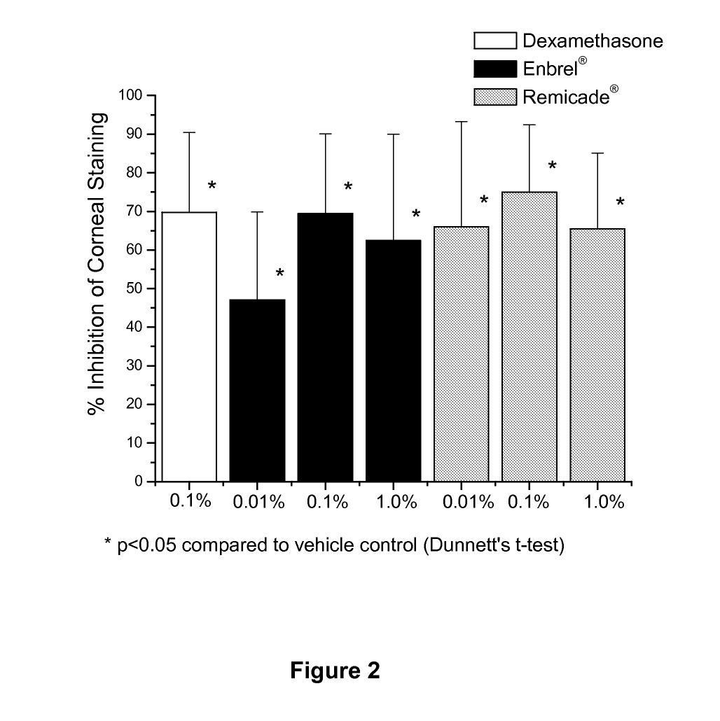

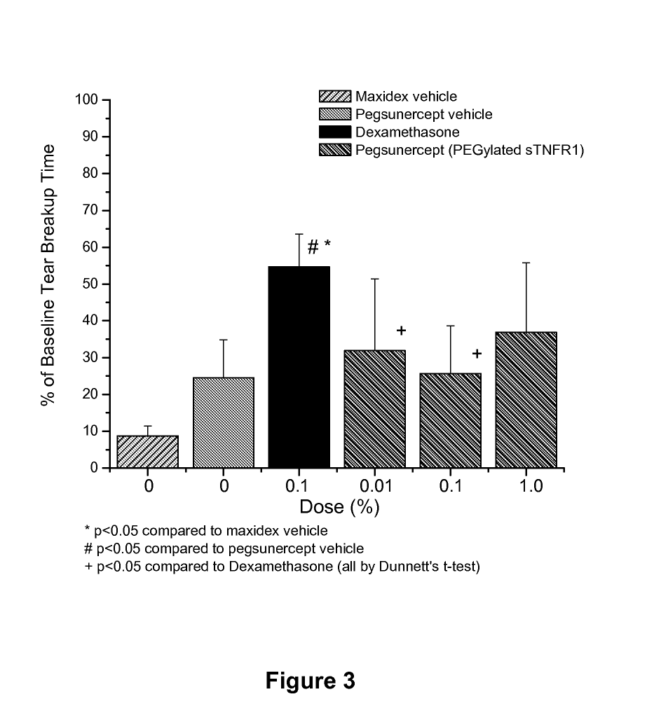

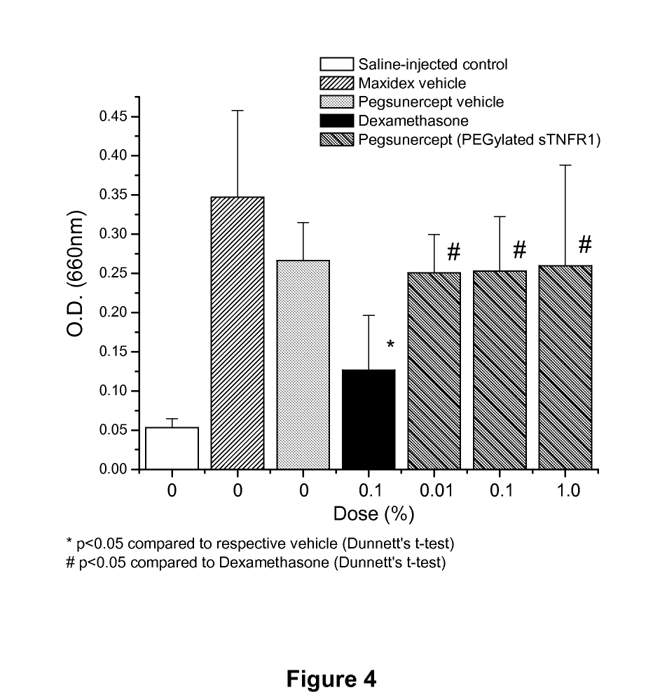

Both etanercept and infliximab effectively increased TBUT and inhibited corneal staining (FIGS. 1 and 2). Efficacy comparable to dexamethasone was achieved for both drugs for each parameter measured. As shown in FIGS. 3 and 4, Pegsunercept was not effective for either TBUT or corneal staining relative to its vehicle over the concentration range studied. FIGS. 3 and 4 also show the vehicle effects on tear breakup time and corneal staining as described in methods. As a TNFα inhibitor, Pegsunercept is less potent than either etanercept or infliximab. The results of these studies indicated that the monomeric soluble TNFα receptor (Pegsunercept) was not effective for treating dry eye at ophthalmically relevant concentrations, whereas the dimeric soluble TNFα receptor (etanercept) and monoclonal anti-TNFα antibody (infliximab) were effective at ophthalmically relevant concentrations.

It should be understood that the foregoing disclosure emphasizes certain specific embodiments of the invention and that all modifications or alternatives equivalent thereto are within the spirit and scope of the invention as set forth in the appended claims.

What is claimed is:

1. A method for the treatment of dry eye which comprises administering to a mammal a composition comprising a pharmaceutically acceptable carrier and a pharmaceutically effective amount of a TNFα inhibitor, wherein the TNFα inhibitor is an antibody that binds TNFα or a soluble dimeric TNFα receptor.

2. The method of claim 1 wherein the pharmaceutically effective amount of the TNFα inhibitor is 0.001-1.0% (w/w).

3. The method of claim 1 wherein the pharmaceutically effective amount of the TNFα inhibitor is 0.01-1.0% (w/w).

4. The method of claim 1 wherein the composition is topically administered to the eye.

5. The method of claim 1 wherein the dry eye is associated with refractive surgery.

6. The method of claim 1 wherein the TNFα inhibitor is etanercept.

7. The method of claim 1 wherein the TNFα inhibitor is infliximab.
